SEQUENCE LISTING (33 sequences)

SEQ ID NO: 1 - protein, 147 aa, Homo sapiens
AVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 2 - protein, 147 aa, Homo sapiens
AVLPQEEEGAGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 3 - protein, 70 aa, Homo sapiens
AVLPQEEEGSGGGQLVTEVTKKEDSCQLGYSAGPCMGMTSRYFYNGTSMACETFQYGGCMGNGNNFVTEK

SEQ ID NO: 4 - protein, 66 aa, Homo sapiens
TVAACNLPIVRGPCRAFIQLWAFDAVKGKCVLFPYGGCQGNGNKFYSEKECREYCGVPGDGDEELL

SEQ ID NO: 5 - protein, 19 aa, Homo sapiens
VPSTPPTPSPSTPPTPSPS

SEQ ID NO: 6 - protein, 101 aa, Homo sapiens
CCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKS

SEQ ID NO: 7 - protein, 131 aa, Homo sapiens
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 8 - protein, 6 aa, Homo sapiens
VPPPPP

SEQ ID NO: 9 - protein, 101 aa, Homo sapiens
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKS

SEQ ID NO: 10 - protein, 131 aa, Homo sapiens; identical to SEQ ID NO: 7

SEQ ID NO: 11 - protein, 58 aa, Homo sapiens
ESPKAQASSVPTAQPQAEGSLAKATTAPATTRNTGRGGEEKKKEKEKEEQEERETKTP

SEQ ID NO: 12 - protein, 108 aa, Homo sapiens
ECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREP

SEQ ID NO: 13 - protein, 117 aa, Homo sapiens
AAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 14 - protein, 107 aa, Homo sapiens
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 15 - protein, 108 aa, Homo sapiens
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 16 - protein, 110 aa, Homo sapiens
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 17 - protein, 15 aa, Homo sapiens
EPKSCDKTHTCPPCP

SEQ ID NO: 18 - protein, 11 aa, Artificial Sequence
  Modified human IgG1 hinge sequence
SDKTHTCPPCP

SEQ ID NO: 19 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 20 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 229 aa, Homo sapiens
MSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 12 aa, Homo sapiens
ERKCCVECPPCP

SEQ ID NO: 23 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 24 - protein, 107 aa, Homo sapiens
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 26 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 27 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 28 - protein, 12 aa, Homo sapiens
ESKYGPPCPSCP

SEQ ID NO: 29 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 30 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 112 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVP

SEQ ID NO: 32 - protein, 106 aa, Homo sapiens
DQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPK

SEQ ID NO: 33 - protein, 131 aa, Homo sapiens
GVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

(19) United States
(12) Patent Application Publication
(10) Pub. No.: US 2022/0242932 A1
(43) Pub. Date: Aug. 4, 2022

(54) ULINASTATIN POLYPEPTIDES FOR TREATING DISEASES

(71) Applicant: DiaMedica USA Inc., Minneapolis, MN (US)

(72) Inventors: Rick PAULS, Minneapolis, MN (US); David WAMBEKE, Edina, MN (US); Sydney GILMAN, Naples, FL (US)

(21) Appl. No.: 17/586,238

(22) Filed: Jan. 27, 2022

Related U.S. Application Data
(60) Provisional application No. 63/143,403, filed on Jan. 29, 2021.

(51) Int. Cl. C07K 14/81; A61P 1/18; A61P 29/00; C07K 14/76

(52) U.S. Cl. CPC C07K 14/8114; A61P 1/18; A61K 38/00; C07K 14/76; A61P 29/00

(57) ABSTRACT

Provided are methods and compositions comprising ulinastatin polypeptides for treating diseases associated with elevated neutrophil elastase (NE), including NE-associated lung and skin diseases.

### CROSS-REFERENCE TO RELATED APPLICATIONS

This application claims priority under 35 U.S.C. § 119 (e) to U.S. Provisional Application No. 63/143,403, filed Jan. 29, 2021, which is incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is DIAM_041_01US_ST25.txt. The text file is about 29 KB, was created on Ja. 24, 2022, and is being submitted electronically via EFS-Web

### BACKGROUND

### Technical Field

Embodiments of the present disclosure include methods and compositions comprising ulinastatin polypeptides for treating diseases associated with elevated neutrophil elastase (NE), including NE-associated lung and skin diseases.

### Description of the Related Art

Ulinastatin (also urinary-trypsin inhibitor) is a glycoprotein proteinase inhibitor derived from human urine which inhibits the activity of trypsin, chymotrypsin, lactate, lipase, hyaluronidase, and various pancreatic enzymes.

Highly-purified ulinastatin has been used clinically for the treatment of acute pancreatitis, chronic pancreatitis, Stevens-Johnson syndrome, burns, septic shock, toxic epidermal necrolysis (TEN), and other diseases. However, large quantities of ulinastatin are required because it is a serpin, a potent protease inhibitor that reacts irreversibly with the active site of the protease and is thus typically consumed in a 1:1 stoichiometry with its target. This property, coupled with the relatively low in vivo exposures achieved after systemic dosing, creates challenges in generating therapeutics from the native ulinastatin protein.

Neutrophil elastase (NE) is a serine proteinase that is secreted by neutrophils and macrophages during inflammation. Excess activity of neutrophil elastase and similar proteases has been reported to cause tissue damage and to alter the remodeling process in many lung conditions such as pneumonia, respiratory distress, and acute lung injury (Polverino et al., Chest. 152(2):249-262, 2017), and skin conditions such as bullous pemphigoid (Liu et al., J Clin Invest. 105(1):113-123, 2000).

There is a need in the art for ulinastatin polypeptides having improved characteristics related to their recombinant production and/or therapeutic utility, and also for expanding the clinical utility of ulinastatin polypeptides to other diseases.

### BRIEF SUMMARY

Embodiments of the present disclosure include methods of treating, ameliorating the symptoms of, or inhibiting the progression of, a neutrophil elastase (NE)-associated disease in a subject in need thereof, comprising administering to the subject a pharmaceutical composition comprising a ulinastatin polypeptide.

In certain embodiments, the NE-associated disease comprises a lung disease. In some embodiments, the lung disease is selected from one or more of alpha-1 antitrypsin (A1AT) deficiency including complications thereof, cystic fibrosis, pneumonia, acute lung injury (ALI), acute respiratory distress syndrome (ARDS), chronic obstructive pulmonary disease (COPD), bronchiectasis, and virus-induced lung injury optionally associated with coronavirus (e.g., Covid-19) or influenza infection, including combinations thereof. In some embodiments, the NE-associated disease is alpha-1 antitrypsin (A1AT)-deficiency including complications thereof.

In certain embodiments, the subject has reduced levels of serum A1AT, optionally about or less than about 80% of normal serum level of A1AT, about or less than about 60% of normal serum level of A1AT, about or less than about 40% of normal serum level of A1AT), or about 10-15% of normal serum level of A1AT.

In some embodiments, the subject has an A1AT phenotype/genotype optionally selected from PiMS, PiSS, PiMZ, PiSZ, and PiZZ. In certain embodiments, the subject has an A1AT genotype selected from a deficiency A1AT allele and a null A1AT allele. In specific embodiments, the A1AT deficiency allele is an E342K allele (Z allele) or an E264V allele (S allele).

Some embodiments comprise the steps of:
(a) determining serum A1AT levels and/or A1AT phenotype/genotype in the subject; and
(b) administering the pharmaceutical composition to the subject if the subject has an A1AT phenotype/genotype and/or reduced serum levels of A1AT levels relative to normal.

In certain embodiments, the A1AT phenotype/genotype is selected from PiMS, PiSS, PiMZ, PiSZ, and PiZZ; wherein the A1AT genotype comprises a deficiency A1AT allele or a null A1AT allele; and/or wherein the serum A1AT levels are about or less than about 80%, 60%, 40%, 20%, 15%, or 10% of normal serum level of A1AT. In some embodiments, the A1AT deficiency allele is an E342K allele (Z allele) or an E264V allele (S allele).

In certain embodiments, the complications of A1AT-deficiency include any one or more of asthma, bronchiectasis, cirrhosis, COPD, respiratory failure, and vasculitis. In certain embodiments, the subject has any one or more of shortness of breath (optionally on exertion and later at rest), wheezing, sputum production, or recurrent respiratory infections.

Particular embodiments comprise administering the pharmaceutical composition comprising the ulinastatin polypeptide in combination with an additional therapeutic agent for treating A1AT-deficiency, optionally an RNA interference (RNAi) agent directed against a disease-associated A1AT mutant, A1AT augmentation therapy, a bronchodilator, an inhaled steroid, an antibiotic agent, or an anti-viral agent.

In certain embodiments, the NE-associated disease is cystic fibrosis, and wherein the subject has mutation in the gene cystic fibrosis transmembrane conductance regulator (CFTR). In some embodiments, the NE-associated disease comprises a skin or mucous membrane disease. In particular embodiments, the skin or mucous membrane disease is Behcet's Disease, pemphigus vulgaris, pemphigus foliaceus, bullous pemphigoid, and Kawasaki disease.

In some embodiments, the NE-associated disease is an autoimmune disease, for example, an autoimmune disease selected from myasthenia gravis, warm autoimmune hemolytic anemia, thyroid eye disease, idiopathic thrombocytopenic purpura, chronic inflammatory demyelinating polyneuropathy, neuromyelitis optica, Guillain-Barre Syndrome, and PLA2R+ membranous nephropathy

In certain embodiments, the subject has increased NE levels or activity relative to a reference standard or healthy control.

Some embodiments comprise the steps of:
(a) determining NE levels or activity in a sample from the subject; and
(b) administering the pharmaceutical composition to the subject if the sample has increased NE levels or activity relative to a reference standard or healthy control.

In some embodiments, the sample is a sputum sample (optionally comprising mucus of the lower airways), a blood or serum sample, or a skin or mucous membrane sample. In some embodiments, the sputum sample comprises airway neutrophils, and the method comprises determining levels or activity of surface-bound NE in the sample. In some embodiments, the method comprises determining levels or activity of free NE in the sample.

In some embodiments, the ulinastatin polypeptide is a recombinant ulinastatin polypeptide or a urinary-derived ulinastatin polypeptide. In some embodiments, the ulinastatin polypeptide comprises, consists, or consists essentially of SEQ ID NO: 2, and comprises an N-linked glycan at residue at residue N45 and an O-linked glycan at residue T17. In some embodiments, the ulinastatin polypeptide comprises, consists, or consists essentially of an amino acid sequence selected from Table U1, or an active variant or fragment thereof that has at least one ulinastatin activity.

In some embodiments, the ulinastatin polypeptide comprises, consists, or consists essentially of:
(a) SEQ ID NO:1 or a variant of SEQ ID NO: 1 that has at least one at least one modification to an O-linked glycosylation and at least one ulinastatin activity;
(b) a fragment of SEQ ID NO: 1 or (a) that has at least one ulinastatin activity; or
(c) a variant of (a) or (b) that is at least 80, 85, 90, 95, 96, 97, 98, or 99% identical to (a) or (b) and has at least one ulinastatin activity.

In some embodiments, (a) comprises a substitution or deletion at the O-linked glycosylation site of residues 8-11 of SEQ ID NO: 1, optionally a substitution or deletion at residue S10 of SEQ ID NO: 1, optionally an S10A substitution, or (b) comprises, consists, or consists essentially of about 20, 25, 30, 35, 40, 45, 50, 55, 60, 65, 70, 75, 80, 85, 90, 95, 100, 105, 110, 115, 120, 125, or 130 contiguous amino acids of SEQ ID NO:1 or 2.

In some embodiments, (b) comprises, consists, or consists essentially of about 20-130, 20-120, 20-110, 20-100, 20-90, 20-80, 20-70, 20-60, 20-50, 20-40, 20-30, 30-130, 30-120, 30-110, 30-100, 30-90, 30-80, 30-70, 30-60, 30-50, 30-40, 40-130, 40-120, 40-110, 40-100, 40-90, 40-80, 40-70, 40-60, 40-50, 50-130, 50-120, 50-110, 50-100, 50-90, 50-80, 50-70, 50-60, 60-130, 60-120, 60-110, 60-100, 60-90, 60-80, 60-70, 70-130, 70-120, 70-110, 70-100, 70-90, 70-80, 80-130, 80-120, 80-110, 80-100, 80-90, 90-130, 90-120, 90-110, 90-100, 100-130, 100-120, or 100-110 contiguous amino acids of SEQ ID NO: 1 or 2.

In some embodiments, (b) comprises, consists, or consists essentially of Domain 1 of SEQ ID NO: 1 or 2. In some embodiments, (b) comprises, consists, or consists essentially of Domain 2 of SEQ ID NO: 1 or 2.

In some embodiments, the ulinastatin polypeptide comprises, consists, or consists essentially of an amino acid sequence that is at least 80, 85, 90, 95, 96, 97, 98, 99, or 100% identical to SEQ ID NO: 2 (UTIΔCS), which retains a substitution at position S10 of SEQ ID NO: 2, optionally a S10A substitution. In some embodiments, the ulinastatin polypeptide has an N-linked glycan at residue at residue N45 and an O-linked glycan at residue T17, the residues being defined by SEQ ID NO: 1 or 2.

In some embodiments, the ulinastatin polypeptide is fused to an Fc region, to form a ulinastatin-Fc fusion polypeptide, wherein the ulinastatin-Fc fusion polypeptide has at least one ulinastatin activity. In some embodiments, the Fc region comprises, consists, or consists essentially of CH2 region, CH3 region, CH4 region, and/or hinge region(s) from a IgA, IgD, IgE, IgG, or IgM immunoglobulin heavy chain. In some embodiments, the Fc region comprises, consists, or consists essentially of one or more of the human Fc region amino acid sequences of Table F1, including variants, fragments, homologs, orthologs, paralogs, and combinations thereof. In some embodiments, the ulinastatin-Fc fusion polypeptide comprises a modified Fc region which has at least one altered effector function and/or pharmacokinetic (PK) characteristic relative to a wild-type Fc region.

In some embodiments, the at least one ulinastatin activity comprises a protease inhibitor activity, optionally a neutrophil elastase (NE)-inhibitor activity or a trypsin inhibitor activity, and optionally an anti-inflammatory activity. In some embodiments, the at least one ulinastatin activity is a trypsin inhibitor activity, and wherein the ulinastatin polypeptide has a specific activity as a trypsin inhibitor of about or at least about 1000-3000 U/mg, or about or at least about 1000, 1100, 1200, 1300, 1400, 1500, 1600, 1700, 1800, 1900, 2000, 2100, 2200, 2300, 2400, 2500, 2600, 2700, 2800, 2900, or 3000 U/mg, wherein one unit (U) is an amount of the ulinastatin polypeptide that inhibits the activity of 2 μg trypsin by 50%. In some embodiments, the at least one ulinastatin activity is an NE-inhibitor activity, and the ulinastatin polypeptide has an IC₅₀ in an in vitro assay of neutrophil elastase activity of about 10 μg/ml or lower value, optionally an IC₅₀ of about 10, 9, 8, 7, 6, 5, 4, 3, 2, or 1 μg/ml or lower value, including all ranges and integers in between, optionally an IC₅₀ of about 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-9, 3-7, 3-6, 3-5, 3-4 10 μg/ml. In some embodiments, the ulinastatin polypeptide has an IC₅₀ in an in vitro assay of neutrophil elastase activity of about 100 nM or lower value, optionally an IC₅₀ of about 100, 90, 80, 70, 60, 50, 40, 30, 20, or 10 nM or lower value, including all ranges and integers in between, optionally an IC₅₀ of about 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, or 40-50 nM

In some embodiments, the pharmaceutical composition has a purity of at least about 80%, 85%, 90%, 95%, 98%, or 99% on a protein basis or a weight-weight basis and is substantially aggregate-free, optionally less than about 10, 9, 8, 7, 6, or 5% aggregated, and wherein the composition is substantially endotoxin-free. In some embodiments, the pharmaceutical composition has less than about 1 EU endotoxin/mg protein, less that about 100 ng host cell protein/mg protein, less than about 10 μg host cell DNA/mg protein, and/or greater than about 95% single peak purity by SEC-HPLC.

Certain embodiments comprise administering the pharmaceutical composition to the subject by intravenous (IV), subcutaneous (SC), inhalatory, topical, or oral administration. Certain embodiments comprise administering the pharmaceutical composition to the subject with a lung disease by inhalatory administration, optionally with a nebulizer, a metered-dose inhaler, or a dry powdered inhaler. Some embodiments comprise administering the pharmaceutical composition to the subject with a skin or mucous membrane disease by topical or oral administration, optionally as an oral mouthwash.

In some embodiments, the pharmaceutical composition comprises the ulinastatin polypeptide at a dosage of about 50,000 U/kg to about 1,000,000 U/kg, including all integers and ranges in between.

In some embodiments, administration of the pharmaceutical composition reduces NE levels or activity in the subject, optionally by about or at least about 10, 20, 30, 40, 50, 60, 70, 80, 90, 100, 200, 300, 400, 500, 600, 700, 800, 900, 1000% or more relative to a control or reference, optionally as measured over a period of about 1, 2, 3, 4, 5, 6, 7, 8, 9, 10, 11, 12, 13, 14, 15, 16, 17, 18, 19, 20, 21, 22, 23, or 24 months or more. In some embodiments, administration of the pharmaceutical composition increase the life expectancy of the subject in need thereof, optionally by about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more years.

In some embodiments, the subject has a lung disease and administration of the pharmaceutical composition improves respiratory function in the subject by about or at least about 10, 20, 30, 40, 50, 60, 70, 80, 90, 100, 200, 300, 400, 500, 600, 700, 800, 900, 1000% or more relative to a control or reference, optionally as measured over a period of about 1, 2, 3, 4, 5, 6, 7, 8, 9, 10, 11, 12, 13, 14, 15, 16, 17, 18, 19, 20, 21, 22, 23, or 24 months or more. In some embodiments, the improved respiratory function is selected from one or more of increased expiration time, increased inspiration time, decreased peak expiratory flow, decreased peak inspiratory flow, decreased respiratory minute volume (RMV), and decreased respiratory rate.

In some embodiments, the subject has a skin or mucous membrane disease and administration of the pharmaceutical composition reduces skin or mucous membrane lesions or ulcers in the subject by about or at least about 10, 20, 30, 40, 50, 60, 70, 80, 90, 100, 200, 300, 400, 500, 600, 700, 800, 900, 1000% or more relative to a control or reference, optionally as measured over a period of about 1, 2, 3, 4, 5, 6, 7, 8, 9, 10, 11, 12, 13, 14, 15, 16, 17, 18, 19, 20, 21, 22, 23, or 24 months or more.

### BRIEF DESCRIPTION OF THE DRAWINGS

FIG. 1 shows that recombinant ulinastatin (DM300) has significant inhibitory activity in an in vitro assay of neutrophil elastase activity.

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The practice of the present disclosure will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Protein Science, Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

As used herein, the term “amino acid” is intended to mean both naturally occurring and non-naturally-occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

“Biocompatible” refers to materials or compounds which are generally not injurious to biological functions and which will not result in any degree of unacceptable toxicity, including allergenic and disease states.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The “half-life” of a polypeptide can refer to the time it takes for the polypeptide to lose half of its pharmacologic, physiologic, or other activity, relative to such activity at the time of administration into the serum or tissue of an organism, or relative to any other defined time-point. “Half-life” can also refer to the time it takes for the amount or concentration of a polypeptide to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and ranges in between e.g., 1.5, 1.6, 1.7. 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18% , 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts, and with respect to ulinastatin is used interchangeably with protein, polypeptide, or peptide. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass the ulinastatin proteins described herein, or sequences that have deletions from, additions to, and/or substitutions of one or more amino acid of the ulinastatin proteins. In certain embodiments, the polypeptide is a “recombinant” polypeptide, which is produced by recombinant cell that comprises one or more recombinant DNA molecules, which are typically made of heterologous polynucleotide sequences or combinations of polynucleotide sequences that would not otherwise be found in the cell.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent (e.g., ulinastatin polypeptide) in a composition may be specifically defined. For instance, certain compositions may comprise an agent that is at least 70, 75 80, 85, 90, 91, 92, 93, 94, 95, 96, 97, 98, 99, or 100% pure (for example, on a protein basis), including all decimals and ranges in between, as measured, for example, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent (e.g., ulinastatin polypeptide) provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCI (with or without NaP). In specific embodiments, solubility is measured at relatively lower pH (e.g., pH 6.0) and relatively higher salt (e.g., 500 mM NaCl and 10 mM NaP). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied mutatis mutandis to every other embodiment unless expressly stated otherwise.

Embodiments of the present disclosure relate to methods of treating, ameliorating the symptoms of, or inhibiting the progression of, a neutrophil elastase (NE)-associated disease in a subject in need thereof, comprising administering to the subject a pharmaceutical composition comprising a ulinastatin polypeptide. Neutrophil elastase (NE; UniProt: P08246)) is a serine protease that hydrolyzes proteins within neutrophil lysosomes (azurophil granules) and extracellular matrix (ECM) proteins upon release from activated neutrophils. It has been shown to play a role in degenerative and inflammatory diseases by its proteolysis of collagen-IV and elastin of the ECM, among other mechanisms. Thus, an “NE-associated disease” includes a disease or condition associated with aberrant or elevated expression or activity of neutrophil elastase relative to a reference standard or healthy control. Human neutrophil elastase is coded by the ELANE gene on chromosome 19.

In certain embodiments, the NE-associated disease comprises a lung disease (see, for example, Sandhaus and Turino, COPD. 10 Suppl 1:60-3, 2013; and Polverino et al., Chest. 152(2):249-262, 2017). Examples of NE-associated lung diseases include alpha-1 antitrypsin (A1AT) deficiency and complications thereof, cystic fibrosis, pneumonia, acute lung injury (ALI), acute respiratory distress syndrome (ARDS), chronic obstructive pulmonary disease (COPD), bronchiectasis, and virus-induced lung injury. In specific embodiments, the virus-induced lung injury is associated with a coronavirus infection, for example, COVID-19 associated with SARS-Cov2 infection. In some embodiments, the virus-induced lung injury is associated with influenza virus infection.

In specific embodiments, the NE-associated disease is alpha-1 antitrypsin (A1AT) deficiency, or A1AD, a genetic disorder that often results in lung disease or liver disease. A1AD is caused by mutations in the SERPINA1 gene, which encodes MAT protein, a protease inhibitor that belongs to the serpin superfamily. A1AT is produced in the liver, and one of its functions is to protect the lungs from neutrophil elastase, an enzyme that can disrupt connective tissue.

Normal blood or serum levels of A1AT vary depending on the analytical method, but are typically around 1.0-2.7 g/l or about 1.26-2.26 g/L (SI Units) or about 126-226 mg/dL (Conventional units). Mutations in the SERPINA1 gene lead to reduced levels of blood or serum MAT. Thus, in certain embodiments, a subject has reduced levels of blood or serum A1AT, for example, about or less than about 80% of normal serum level of A1AT, about or less than about 60% of normal serum level of A1AT, about or less than about 40% of normal serum level of A1AT, or about or less than about 10-15% of normal serum level of A1AT.

In certain embodiments, the subject has an A1AT phenotype/genotype, for example, selected from PiMS, PiSS, PiMZ, PiSZ, and PiZZ. In some embodiments, the subject has a PiMS phenotype/genotype and about or less than about 80% of normal serum level of A1AT. In some embodiments, the subject has a PiSS phenotype/genotype and about or less than about 60% of normal serum level of A1AT. In some embodiments, the subject has a PiMZ phenotype/genotype and about or less than about 60% of normal serum level of A1AT. In certain embodiments, the subject has a PiSZ phenotype/genotype and about or less than about 40% of normal serum level of A1AT. In particular embodiments, the subject has a PiZZ phenotype/genotype and about or less than about 10-15% of normal serum level of A1AT. In some embodiments, the subject has an A1AT genotype selected from a deficiency A1AT allele and a null A1AT allele (for example, an RNA null that produces no A1AT RNA transcript, or a protein null that produces no A1AT protein). Numerous deficiency A1AT alleles are known in the art, predominant examples of which include the E342K allele (or Z allele) and the E264V allele (or S allele).

In certain embodiments, the subject has one or more complications and/or symptoms of A1AT-deficiency or other NE-associated lung disease. Examples of complications of A1AT-deficiency include asthma, bronchiectasis, cirrhosis, COPD, respiratory failure, and vasculitis. Exemplary symptoms of A1AT-deficiency or other lung disease include shortness of breath (for example, on exertion and later at rest), wheezing, sputum production, and recurrent respiratory infections.

Certain embodiments include the steps of determining serum A1AT levels and/or A1AT phenotype/genotype in the subject, and administering the pharmaceutical composition (comprising a ulinastatin polypeptide) to the subject if the subject has an A1AT phenotype/genotype and/or reduced serum levels of A1AT levels relative to normal. Methods of determining serum A1AT levels and/or A1AT phenotype/genotype are known in the art (see, for example, Silverman and Sandhaus, N Engl J Med. 360(26):2749-57, 2009), and include protein electrophoresis assays, nephelometric or immunoturbidimetric assays, enzyme-linked-immuno-sorbent assays (ELISAs), and radial immunodiffusion methods. Certain embodiments include administering the pharmaceutical composition if the subject is determined to have a A1AT phenotype/genotype selected from PiMS, PiSS, PiMZ, PiSZ, and PiZZ; if the subject is determined to have a deficiency A1AT allele (for example, Z allele, S allele) or a null A1AT allele; and/or if the subject is determined to have serum A1AT levels that are about or less than about 80%, 60%, 40%, 20%, 15%, or 10% of normal serum level of A1AT.

Particular embodiments include combination therapies for treating A1AT-deficiency. For instance, certain embodiments comprising administering the pharmaceutical composition comprising the ulinastatin polypeptide in combination with an additional therapeutic agent for treating A1AT-deficiency, for example, an RNA interference (RNAi) agent directed against a disease-associated A1AT mutant (for example, ARO-ATT), A1AT augmentation therapy, a bronchodilator, an inhaled steroid, an antibiotic agent, or an anti-viral agent.

In certain embodiments, the NE-associated lung disease is cystic fibrosis, an autosomal recessive genetic disorder associated with mutations in the gene cystic fibrosis transmembrane conductance regulator (CFTR). Neutrophil elastase is a key risk factor for the severity of cystic fibrosis lung disease (see, for example, Dittrich et al., Eur Respir J. 51(3):1701910, 2018). Thus, in certain embodiments, the subject has cystic fibrosis and mutations in the gene CFTR.

In certain embodiments, the NE-associated disease comprises a skin or mucous membrane disease (see, for example, Liu et al., J Clin Invest. 105(1):113-123, 2000). Examples of skin or mucous membrane diseases include Behcet's Disease, pemphigus vulgaris, pemphigus foliaceus, bullous pemphigoid, and Kawasaki disease.

In certain embodiments, the NE-associated disease comprises an autoimmune disease. Examples of autoimmune diseases include myasthenia gravis, warm autoimmune hemolytic anemia, thyroid eye disease, idiopathic thrombocytopenic purpura, chronic inflammatory demyelinating polyneuropathy, neuromyelitis optica, Guillain-Barre Syndrome, and PLA2R+ membranous nephropathy.

In certain embodiments, the subject has increased neutrophil elastase (NE) levels and/or activity relative to a reference standard or healthy control. In some embodiments, the subject has increased NE levels in blood/serum, increased NE levels in sputum, and/or increased NE levels in skin or mucous membrane(s). For example, in some embodiments, the NE levels in the subject are increased by about or at least about 10, 20, 30, 40, 50, 60, 70, 80, 90, 100, 200, 300, 400, 500, 600, 700, 800, 900, 1000% or more relative to a reference standard or healthy control. Certain embodiments include the step of determining NE levels and/or activity in a sample from the subject, and administering the pharmaceutical composition (comprising the ulinastatin polypeptide) to the subject if the sample has increased NE levels and/or activity relative to a reference standard or healthy control. In some embodiments, the sample is a sputum sample (for example, comprising mucus of the lower airways), a blood or serum sample, or a skin or mucous membrane sample. In specific embodiments, the sputum sample comprises airway neutrophils, and the method comprises determining levels or activity of surface-bound NE in the sample. In particular embodiments, the method comprises determining levels or activity of free NE in the sample. Methods for determining NE levels and/or activity are known in the art (see, for example, Shoemark et al., Eur Respir J. 53(6):1900303, 2019).

The methods provided herein include administering a pharmaceutical composition comprising a “ulinastatin polypeptide”. “Ulinastatin” (also referred to as urinary trypsin inhibitor (UTI), H1-30, ASPI, or bikunin) is an acidic glycoprotein with a molecular weight of about 30 kDa by SDS-polyacrylamide gel electrophoresis. Wild-type human ulinastatin is a multivalent Kunitz-type serine protease inhibitor found in human urine and blood, and which includes two Kunitz-type domains. It is expressed as a full-length protein, and processed to the mature, or active form of ulinastatin. In certain embodiments, the ulinastatin polypeptide is recombinant ulinastatin polypeptide or a urinary-derived ulinastatin polypeptide. In specific embodiments, the ulinastatin polypeptide is the mature form of ulinastatin, or a variant or fragment or domain thereof.

Exemplary polypeptide sequences for mature ulinastatin and domains thereof are provided in Table U1.

TABLE U1
Exemplary Ulinastatin Polypeptide Sequences

| | | SEQ ID |
|---|---|---|
| Description | Sequence | NO: |
| WT Mature | AVLPQEEEGSGGGQLVTEVTKKEDSCQLGYSAGPCMGMTSRYFYNGTSM | 1 |
| Human | ACETFQYGGCMGNGNNFVTEKECLQTCRTVAACNLPIVRGPCRAFIQLW | |
| Ulinastatin | AFDAVKGKCVLFPYGGCQGNGNKFYSEKECREYCGVPGDGDEELLRFSN | |
| Mature Human | AVLPQEEEG [symbol] GGGQLVTEVTKKEDSCQLGYSAGPCMGMTSRYFYNGTSM | 2 |
| Ulinastatin | ACETFQYGGCMGNGNNFVTEKECLQTCRTVAACNLPIVRGPCRAFIQLW | |
| S10A Mutation | AFDAVKGKCVLFPYGGCQGNGNKFYSEKECREYCGVPGDGDEELLRFSN | |
| Domain 1 | AVLPQEEEGSGGGQLVTEVTKKEDSCQLGYSAGPCMGMTSRYFYNGTSM | 3 |
| | ACETFQYGGCMGNGNNFVTEK | |
| Domain 2 | TVAACNLPIVRGPCRAFIQLWAFDAVKGKCVLFPYGGCQGNGNKFYSEK | 4 |
| | ECREYCGVPGDGDEELL | |

Thus, in some embodiments, the ulinastatin polypeptide comprises, consists, or consists essentially of an amino acid sequence selected from Table U1, or an active variant or fragment thereof that has at least one ulinastatin activity. In some embodiments, the ulinastatin polypeptide comprises, consists, or consists essentially of:
(a) SEQ ID NO:1 or a variant of SEQ ID NO: 1 that has at least one at least one modification to an O-linked glycosylation and at least one ulinastatin activity;
(b) a fragment of SEQ ID NO: 1 or (a) that has at least one ulinastatin activity; or
(c) a variant of (a) or (b) that is at least 80, 85, 90, 95, 96, 97, 98, or 99% identical to (a) or (b) and has at least one ulinastatin activity.

Certain ulinastatin polypeptides have a modified O-linked glycosylation site. Here, serine 10 has a chondroitin sulfate (CS) chain attached at a well-conserved Glu-Gly-Ser-Gly (SEQ ID NO: 5) O-linked glycosylation site. The CS chain is relatively short (Mwt˜8000) with 12-18 disaccharide repeats (GlcUA 1,3-GalNac1,4-) and a conventional linkage region (GlcUA 1-3Gal 1-3Gal 1- 4Xyl 1)-O-Ser. About 30% of the GaINAc, usually those near the linkage region, are sulfated at C-4 hydroxyl groups. CS chains synthesized during inflammations are shorter with decreased sulfation. Thus, in some instances, a ulinastatin polypeptide comprises at least one substitution and/or deletion at one or more of the Glu-Gly-Ser-Gly (SEQ ID NO: 5) residues of SEQ ID NO:1, which reduces glycosylation at the O-linked glycosylation site. In specific embodiments, a ulinastatin polypeptide comprises a substitution or deletion at position S10 of SEQ ID NO: 1, for example, an S10A substitution (see SEQ ID NO: 2) or other conservative substitution. In some embodiments, the ulinastatin polypeptide comprises a substitution or deletion at the O-linked glycosylation site of residues 8-11 of SEQ ID NO: 1, optionally a substitution or deletion at residue S10 of SEQ ID NO: 1, optionally an S10A substitution, or wherein (b) comprises, consists, or consists essentially of about 20, 25, 30, 35, 40, 45, 50, 55, 60, 65, 70, 75, 80, 85, 90, 95, 100, 105, 110, 115, 120, 125, or 130 contiguous amino acids of SEQ ID NO:1 or 2.

In some instances, a ulinastatin polypeptide comprises, consists, or consists essentially of about 20, 25, 30, 35, 40, 45, 50, 55, 60, 65, 70, 75, 80, 85, 90, 95, 100, 105, 110, 115, 120, 125, or 130 contiguous amino acids of SEQ ID NO:1 or 2. In some instances, a ulinastatin polypeptide comprises, consists, or consists essentially of about 20-130, 20-120, 20-110, 20-100, 20-90, 20-80, 20-70, 20-60, 20-50, 20-40, 20-30, 30-130, 30-120, 30-110, 30-100, 30-90, 30-80, 30-70, 30-60, 30-50, 30-40, 40-130, 40-120, 40-110, 40-100, 40-90, 40-80, 40-70, 40-60, 40-50, 50-130, 50-120, 50-110, 50-100, 50-90, 50-80, 50-70, 50-60, 60-130, 60-120, 60-110, 60-100, 60-90, 60-80, 60-70, 70-130, 70-120, 70-110, 70-100, 70-90, 70-80, 80-130, 80-120, 80-110, 80-100, 80-90, 90-130, 90-120, 90-110, 90-100, 100-130, 100-120, or 100-110 contiguous amino acids of SEQ ID NO: 1 or 2.

In some embodiments, a ulinastatin polypeptide comprises, consists, or consists essentially of Domain 1 of SEQ ID NO: 1 or 2 or an active variant thereof. In certain embodiments, a ulinastatin polypeptide comprises, consists, or consists essentially of Domain 2 of SEQ ID NO: 1 or 2 or an active variant thereof.

In certain embodiments, the ulinastatin polypeptide has an N-linked glycan at residue at residue N45 and/or a non-natural O-linked glycan at residue T17, the residues being defined by SEQ ID NO: 1 or 2.

In some embodiments, the ulinastatin polypeptide comprises, consists, or consists essentially of an amino acid sequence that is at least 80, 85, 90, 95, 96, 97, 98, 99, or 100% identical to SEQ ID NO: 2, which retains a substitution at position S10 of SEQ ID NO: 2, optionally a S10A substitution. In specific embodiments, the ulinastatin polypeptide comprises, consists, or consists essentially of SEQ ID NO: 2, and comprises an N-linked glycan at residue N45 and/or an O-linked glycan at residue T17.

In certain embodiments, the ulinastatin polypeptide is fused to an Fc region, to form a ulinastatin-Fc fusion polypeptide, which has at least one ulinastatin activity. In certain embodiments, Fc region comprises, consists, or consists essentially of CH2 region, CH3 region, CH4 region, and/or hinge region(s) from a IgA, IgD, IgE, IgG, or IgM immunoglobulin heavy chain. In some instances, the Fc region is a human Fc region. In some embodiments, an Fc region has high effector function in humans, for example, an IgG1 Fc region or an IgG3 Fc region. In some embodiments, an Fc region has low effector function in humans, for example, an IgG2 Fc region or an IgG4 Fc region.

The amino acid sequences of CH2, CH3, CH4, and hinge regions from exemplary, wild-type human IgA1, IgA2, IgD, IgE, IgG1, IgG2, IgG3, IgG4, and IgM immunoglobulins are shown in Table F1 below.

TABLE F1
Exemplary Fc sequences

| | | SEQ ID |
|---|---|---|
| Name | Sequence | NO: |
| IgA1 hinge | VPSTPPTPSPSTPPTPSPS | 5 |
| IgA1 CH2 | CCHPRLSLHRPALEDLLLGSEANLTCTLTGLRDASGVTFTWTPSSGKSA | 6 |
| | VQGPPERDLCGCYSVSSVLPGCAEPWNHGKTFTCTAAYPESKTPLTATL | |
| | SKS | |
| IgA1 CH3 | GNTFRPEVHLLPPPSEELALNELVTLTCLARGFSPKDVLVRWLQGSQEL | 7 |
| | PREKYLTWASRQEPSQGTTTFAVTSILRVAAEDWKKGDTFSCMVGHEAL | |
| | PLAFTQKTIDRLAGKPTHVNVSVVMAEVDGTCY | |
| IgA2 hinge | VPPPPP | 8 |
| IgA2 CH2 | CCHPRLSLHRPALEDLLLGSEANLTCTLTGLRDASGATFTWTPSSGKSA | 9 |
| | VQGPPERDLCGCYSVSSVLPGCAQPWNHGETFTCTAAHPELKTPLTANI | |
| | TKS | |
| IgA2 CH3 | GNTFRPEVHLLPPPSEELALNELVTLTCLARGFSPKDVLVRWLQGSQEL | 10 |
| | PREKYLTWASRQEPSQGTTTFAVTSILRVAAEDWKKGDTFSCMVGHEAL | |
| | PLAFTQKTIDRLAGKPTHVNVSVVMAEVDGTCY | |
| IgD hinge | ESPKAQASSVPTAQPQAEGSLAKATTAPATTRNTGRGGEEKKKEKEKEE | 11 |
| | QEERETKTP | |
| IgD CH2 | ECPSHTQPLGVYLLTPAVQDLWLRDKATFTCFVVGSDLKDAHLTWEVAG | 12 |
| | KVPTGGVEEGLLERHSNGSQSQHSRLTLPRSLWNAGTSVTCTLNHPSLP | |
| | PQRLMALREP | |
| IgD CH3 | AAQAPVKLSLNLLASSDPPEAASWLLCEVSGFSPPNILLMWLEDQREVN | 13 |
| | TSGFAPARPPPQPRSTTFWAWSVLRVPAPPSPQPATYTCVVSHEDSRTL | |
| | LNASRSLEVSYVTDHGPMK | |
| IgE CH2 | VCSRDFTPPTVKILQSSCDGGGHFPPTIQLLCLVSGYTPGTINITWLED | 14 |
| | GQVMDVDLSTASTTQEGELASTQSELTLSQKHWLSDRTYTCQVTYQGHT | |
| | FEDSTKKCA | |
| IgE CH3 | DSNPRGVSAYLSRPSPFDLFIRKSPTITCLVVDLAPSKGTVNLTWSRAS | 15 |
| | GKPVNHSTRKEEKQRNGTLTVTSTLPVGTRDWIEGETYQCRVTHPHLPR | |
| | ALMRSTTKTS | |
| IgE CH4 | GPRAAPEVYAFATPEWPGSRDKRTLACLIQNFMPEDISVQWLHNEVQLP | 16 |
| | DARHSTTQPRKTKGSGFFVFSRLEVTRAEWEQKDEFICRAVHEAASPSQ | |
| | TVQRAVSVNPGK | |
| IgG1 hinge | EPKSCDKTHTCPPCP | 17 |
| modified | SDKTHTCPPCP | 18 |
| human IgG1 | | |
| hinge | | |
| IgG1 CH2 | APELLGGPSVFLFPPKPKDTLMISRTPEVTCVVVDVSHEDPEVKFNWYV | 19 |
| | DGVEVHNAKTKPREEQYNSTYRVVSVLTVLHQDWLNGKEYKCKVSNKAL | |
| | PAPIEKTISKAK | |
| IgG1 CH3 | GQPREPQVYTLPPSRDELTKNQVSLTCLVKGFYPSDIAVEWESNGQPEN | 20 |
| | NYKTTPPVLDSDGSFFLYSKLTVDKSRWQQGNVFSCSVMHEALHNHYTQ | |
| | KSLSLSPGK | |
| IgG1 heavy | MSDKTHTCPPCPAPELLGGPSVFLFPPKPKDTLMISRTPEVTCVVVDVS | 21 |
| chain | HEDPEVKFNWYVDGVEVHNAKTKPREEQYNSTYRVVSVLTVLHQDWLNG | |
| | KEYKCKVSNKALPAPIEKTISKAKGQPREPQVYTLPPSREEMTKNQVSL | |
| | TCLVKGFYPSDIAVEWESNGQPENNYKTTPPVLDSDGSFFLYSKLTVDK | |
| | SRWQQGNVFSCSVMHEALHNHYTQKSLSLSPGK | |
| IgG2 hinge | ERKCCVECPPCP | 22 |
| IgG2 CH2 | APPVAGPSVFLFPPKPKDTLMISRTPEVTCVVVDVSHEDPEVQFNWYVD | 23 |
| | GVEVHNAKTKPREEQFNSTFRVVSVLTVVHQDWLNGKEYKCKVSNKGLP | |
| | APIEKTISKTK | |
| IgG2 CH3 | GQPREPQVYTLPPSREEMTKNQVSLTCLVKGFYPSDIAVEWESNGQPEN | 24 |
| | NYKTTPPMLDSDGSFFLYSKLTVDKSRWQQGNVFSCSVMHEALHNHYTQ | |
| | KSLSLSPGK | |
| IgG3 hinge | ELKTPLGDTTHTCPRCPEPKSCDTPPPCPRCPEPKSCDTPPPCPRCPEP | 25 |
| | KSCDTPPPCPRCP | |
| IgG3 CH2 | APELLGGPSVFLFPPKPKDTLMISRTPEVTCVVVDVSHEDPEVQFKWYV | 26 |
| | DGVEVHNAKTKPREEQYNSTFRVVSVLTVLHQDWLNGKEYKCKVSNKAL | |
| | PAPIEKTISKTK | |
| IgG3 CH3 | GQPREPQVYTLPPSREEMTKNQVSLTCLVKGFYPSDIAVEWESSGQPEN | 27 |
| | NYNTTPPMLDSDGSFFLYSKLTVDKSRWQQGNIFSCSVMHEALHNRFTQ | |
| | KSLSLSPGK | |
| IgG4 hinge | ESKYGPPCPSCP | 28 |
| IgG4 CH2 | APEFLGGPSVFLFPPKPKDTLMISRTPEVTCVVVDVSQEDPEVQFNWYV | 29 |
| | DGVEVHNAKTKPREEQFNSTYRVVSVLTVLHQDWLNGKEYKCKVSNKGL | |
| | PSSIEKTISKAK | |
| IgG4 CH3 | GQPREPQVYTLPPSQEEMTKNQVSLTCLVKGFYPSDIAVEWESNGQPEN | 30 |
| | NYKTTPPVLDSDGSFFLYSRLTVDKSRWQEGNVFSCSVMHEALHNHYTQ | |
| | KSLSLSLGK | |
| IgM CH2 | VIAELPPKVSVFVPPRDGFFGNPRKSKLICQATGFSPRQIQVSWLREGK | 31 |
| | QVGSGVTTDQVQAEAKESGPTTYKVTSTLTIKESDWLGQSMFTCRVDHR | |
| | GLTFQQNASSMCVP | |
| IgM CH3 | DQDTAIRVFAIPPSFASIFLTKSTKLTCLVTDLTTYDSVTISWTRQNGE | 32 |
| | AVKTHTNISESHPNATFSAVGEASICEDDWNSGERFTCTVTHTDLPSPL | |
| | KQTISRPK | |
| IgM CH4 | GVALHRPDVYLLPPAREQLNLRESATITCLVTGFSPADVFVQWMQRGQP | 33 |
| | ISPEKYVTSAPMPEPQAPGRYFAHSILTVSEEEWNTGETYTCVVAHEAL | |
| | PNRVTERTVDKSTGKPTLYNVSLVMSDTAGTCY | |

Thus, in some embodiments, the Fc region in a ulinastatin-Fc fusion polypeptide comprises, consists, or consists essentially of one or more of the human Fc region amino acid sequences of Table F1, including variants, fragments, homologs, orthologs, paralogs, and combinations thereof. Certain illustrative embodiments comprise an Fc region that ranges in size from about 20-50, 20-100, 20-150, 20-200, 20-250, 20-300, 20-400, 50-100, 50-150, 50-200, 50-250, 50-300, 50-400, 100-150, 100-200, 100-250, 100-300, 100-350, 100-400, 200-250, 200-300, 200-350, or 200-400 amino acids in length, and optionally comprises, consists of, or consists essentially of any one or more of the sequences in Table F1. Certain embodiments comprise an Fc region of up to about 50, 60, 70, 80, 90, 100, 110, 120, 130, 140, 150, 160, 170, 180, 190, 200, 210, 220, 230, 240, 250, 300, 350, 400 or more amino acids, which optionally comprises, consists of, or consists essentially of any one or more of the amino acid sequences of Table F1.

Certain ulinastatin Fc fusion polypeptides comprise a modified Fc region, including Fc regions having altered properties or biological activities relative to wild-type Fc region(s). In particular embodiments, a modified Fc region has at least one altered effector function and/or pharmacokinetic (PK) characteristic relative to a wild-type Fc region. Thus in certain embodiments, a fusion polypeptide has a modified Fc region with altered functional properties, such as reduced or enhanced CDC, ADCC, or ADCP activity, reduced or enhanced binding affinity for a specific FcγR, or increased serum half-life. Specific embodiments include modified Fc regions having altered (e.g., increased, decreased) binding to FcRs such as FcγR1, FcγRIIa, FcγRIIb, FcγRIIc, FcγRIIIa, FcγRIIIb, and/or FcRn; pharmacokinetic properties such as stability or half-life, bioavailability, tissue distribution, volume of distribution, concentration, elimination rate constant, elimination rate, area under the curve (AUC), clearance, Cmax, tmax, Cmin, and/or fluctuation; immunogenicity; complement fixation or activation; and/or CDC/ADCC/ADCP-related activities, relative to a corresponding wild-type Fc region. Included are modified Fc regions of human and/or mouse origin.

In certain embodiments, a peptide linker sequence may be employed to separate the ulinastatin polypeptide and the Fc region by a distance sufficient to ensure that each polypeptide folds into its desired secondary and tertiary structures, and/or to facilitate cleavage of the signal peptide from the ulinastatin polypeptide, if desired. Such a peptide linker sequence can be incorporated into a fusion polypeptide using standard techniques well known in the art.

Certain peptide linker sequences may be chosen based on the following exemplary factors: (1) their ability to adopt a flexible extended conformation; (2) their inability to adopt a secondary structure that could interact with functional epitopes on the first and second polypeptides; (3) their physiological stability; and (4) the lack of hydrophobic or charged residues that might react with the polypeptide functional epitopes, or other features. See, e.g., George and Heringa, J Protein Eng. 15:871-879, 2002.

The linker sequence may generally be from 1 to about 200 amino acids in length. Particular linkers can have an overall amino acid length of about 1-200 amino acids, 1-150 amino acids, 1-100 amino acids, 1-90 amino acids, 1-80 amino acids, 1-70 amino acids, 1-60 amino acids, 1-50 amino acids, 1-40 amino acids, 1-30 amino acids, 1-20 amino acids, 1-10 amino acids, 1-5 amino acids, 1-4 amino acids, 1-3 amino acids, or about 1, 2, 3, 4, 5, 6, 7, 8, 9, 10, 11, 12, 13, 14, 15, 16 ,17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more amino acids.

In certain embodiments, a ulinastatin polypeptide (including variants, fragments, and fusion polypeptides) has at least one ulinastatin activity, including protease inhibitor activities and anti-inflammatory activities. In some embodiments, a ulinastatin polypeptide has a specific activity of about or at least about 1000-3000 U/mg, or about or at least about 1000, 1100, 1200, 1300, 1400, 1500, 1600, 1700, 1800, 1900, 2000, 2100, 2200, 2300, 2400, 2500, 2600, 2700, 2800, 2900, or 3000 U/mg, wherein one unit (U) is an amount of the ulinastatin polypeptide that inhibits the activity of 2 μg trypsin by 50%. In some embodiments, the at least one ulinastatin activity comprises a neutrophil elastase (NE)-inhibitor activity. In certain embodiments, the ulinastatin polypeptide has an IC₅₀ in an in vitro assay of neutrophil elastase activity (see Example 1) of about or lower than about 10 μg/ml, for example, about 10, 9, 8, 7, 6, 5, 4, 3, 2, or 1 μg/ml or lower value, including all ranges and integers in between, for example, about 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-9, 3-7, 3-6, 3-5, 3-4 10 μg/ml. In some embodiments, the ulinastatin polypeptide has an IC₅₀ in an in vitro assay of neutrophil elastase activity of about 100 nM or lower value, optionally an IC₅₀ of about 100, 90, 80, 70, 60, 50, 40, 30, 20, or 10 nM or lower value, including all ranges and integers in between, optionally an IC₅₀ of about 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, or 40-50 nM. In certain embodiments, the at least one ulinastatin activity is measured or characterized under physiological conditions, for example, of temperature, salinity, and/or pH.

As noted above, certain embodiments include “variants” of a ulinastatin polypeptide or fusion polypeptide described herein. A “variant” sequence refers to a polypeptide or polynucleotide sequence that differs from a reference sequence by one or more substitutions, deletions (e.g., truncations), additions, and/or insertions. Certain variants thus include fragments of a reference sequence described herein. Variant polypeptides are biologically active, that is, they continue to possess the enzymatic or binding activity of a reference polypeptide. Such variants may result from, for example, genetic polymorphism and/or from human manipulation.

In some instances, a variant comprises one or more “conservative” changes or substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present disclosure and still obtain a functional molecule that encodes a variant or derivative polypeptide with desirable characteristics. When it is desired to alter the amino acid sequence of a polypeptide to create an equivalent, or even an improved, variant or portion of a polypeptide described herein, one skilled in the art will typically change one or more of the codons of the encoding DNA sequence.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their utility.

In making such changes, the hydropathic index of amino acids may be considered. The importance of the hydropathic amino acid index in conferring interactive biologic function on a protein is generally understood in the art (Kyte & Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte & Doolittle, 1982). These values are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred.

It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0 ±1); glutamate (+3.0 ±1); serine (+0.3); asparagine (+0.2); glutamine (+0.2); glycine (0); threonine (−0.4); proline (−0.5 ±1); alanine (−0.5); histidine (−0.5); cysteine (−1.0); methionine (−1.3); valine (−1.5); leucine (−1.8); isoleucine (−1.8); tyrosine (-2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

A variant may also, or alternatively, contain non-conservative changes. In some embodiments, variant polypeptides differ from a native or reference sequence by substitution, deletion or addition of about or fewer than about 10, 9, 8, 7, 6, 5, 4, 3, 2 amino acids, or even 1 amino acid. Variants may also (or alternatively) be modified by, for example, the deletion or addition of amino acids that have minimal influence on the immunogenicity, secondary structure, enzymatic activity, and/or hydropathic nature of the polypeptide.

In certain embodiments, a polypeptide sequence is about, at least about, or up to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, or 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 800, 810, 820, 830, 840, 850, 860, 870, 880, 890, 900, 900, 910, 920, 930, 940, 950, 960, 970, 980, 990, 1000 or more contiguous amino acids in length, including all integers in between, and which may comprise all or a portion of a reference sequence (see, e.g., Table U1, Sequence Listing).

In some embodiments, a polypeptide sequence consists of about or no more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 800, 810, 820, 830, 840, 850, 860, 870, 880, 890, 900, 900, 910, 920, 930, 940, 950, 960, 970, 980, 990, 1000 or more contiguous amino acids, including all integers in between, and which may comprise all or a portion of a reference sequence (see, e.g., Table U1, Sequence Listing).

In certain embodiments, a polypeptide sequence is about 10-1000, 10-900, 10-800, 10-700, 10-600, 10-500, 10-400, 10-300, 10-200, 10-100, 10-50, 10-40, 10-30, 10-20, 20-1000, 20-900, 20-800, 20-700, 20-600, 20-500, 20-400, 20-300, 20-200, 20-100, 20-50, 20-40, 20-30, 50-1000, 50-900, 50-800, 50-700, 50-600, 50-500, 50-400, 50-300, 50-200, 50-100, 100-1000, 100-900, 100-800, 100-700, 100-600, 100-500, 100-400, 100-300, 100-200, 200-1000, 200-900, 200-800, 200-700, 200-600, 200-500, 200-400, or 200-300 contiguous amino acids, including all ranges in between, and comprises all or a portion of a reference sequence (see, e.g., Table U1, Sequence Listing). In certain embodiments, the C-terminal or N-terminal region of any reference polypeptide may be truncated by about 1, 2, 3, 4, 5, 6, 7, 8, 9, 10, 15, 20, 25, 30, 35, 40, 45, 50, 60, 70, 80, 90, or 100 or more amino acids, or by about 10-50, 20-50, 50-100 or more amino acids, including all integers and ranges in between (e.g., 101, 102, 103, 104, 105), so long as the truncated polypeptide retains the binding properties and/or activity of the reference polypeptide (see, e.g., Table U1, Sequence Listing). Typically, the biologically-active fragment has no less than about 1%, about 5%, about 10%, about 25%, or about 50% of an activity of the biologically-active reference polypeptide from which it is derived.

In general, variants will display at least about 30%, 40%, 50%, 55%, 60%, 65%, 70%, 75%, 80%, 85%, 90%, 91%, 92%, 93%, 94%, 95%, 96%, 97%, 98%, 99% similarity or sequence identity or sequence homology to a reference polypeptide sequence (see, e.g., Table U1, Sequence Listing). Moreover, sequences differing from the native or parent sequences by the addition (e.g., C-terminal addition, N-terminal addition, both), deletion, truncation, insertion, or substitution (e.g., conservative substitution) of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids (including all integers and ranges in between) but which retain the properties or activities of a parent or reference polypeptide sequence are contemplated (see, e.g., Table U1, Sequence Listing).

In some embodiments, variant polypeptides differ from reference sequence by at least one but by less than 50, 40, 30, 20, 15, 10, 8, 6, 5, 4, 3 or 2 amino acid residue(s). In certain embodiments, variant polypeptides differ from a reference sequence by at least 1% but less than 20%, 15%, 10% or 5% of the residues. (If this comparison requires alignment, the sequences should be aligned for maximum similarity. “Looped” out sequences from deletions or insertions, or mismatches, are considered differences.)

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (J. Mol. Biol. 48: 444-453, 1970) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (Cabios. 4:11-17, 1989) which has been incorporated into the ALIGN program (version 2.0), using a PAM120 weight residue table, a gap length penalty of 12 and a gap penalty of 4.

The sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength =12 to obtain nucleotide sequences homologous to nucleic acid molecules described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (Nucleic Acids Res. 25: 3389-3402, 1997). When utilizing BLAST and Gapped BLAST programs, the default parameters of the respective programs (e.g., XBLAST and NBLAST) can be used.

In some embodiments, as noted above, polynucleotides and/or polypeptides can be evaluated using a BLAST alignment tool. A local alignment consists simply of a pair of sequence segments, one from each of the sequences being compared. A modification of Smith-Waterman or Sellers algorithms will find all segment pairs whose scores cannot be improved by extension or trimming, called high-scoring segment pairs (HSPs). The results of the BLAST alignments include statistical measures to indicate the likelihood that the BLAST score can be expected from chance alone.

The raw score, S, is calculated from the number of gaps and substitutions associated with each aligned sequence wherein higher similarity scores indicate a more significant alignment. Substitution scores are given by a look-up table (see PAM, BLOSUM).

Gap scores are typically calculated as the sum of G, the gap opening penalty and L, the gap extension penalty. For a gap of length n, the gap cost would be G+Ln. The choice of gap costs, G and L is empirical, but it is customary to choose a high value for G (10-15), e.g., 11, and a low value for L (1-2) e.g., 1.

The bit score, S′, is derived from the raw alignment score S in which the statistical properties of the scoring system used have been taken into account. Bit scores are normalized with respect to the scoring system, therefore they can be used to compare alignment scores from different searches. The terms “bit score” and “similarity score” are used interchangeably. The bit score gives an indication of how good the alignment is; the higher the score, the better the alignment.

The E-Value, or expected value, describes the likelihood that a sequence with a similar score will occur in the database by chance. It is a prediction of the number of different alignments with scores equivalent to or better than S that are expected to occur in a database search by chance. The smaller the E-Value, the more significant the alignment. For example, an alignment having an E value of e⁻¹¹⁷ means that a sequence with a similar score is very unlikely to occur simply by chance. Additionally, the expected score for aligning a random pair of amino acids is required to be negative, otherwise long alignments would tend to have high score independently of whether the segments aligned were related. Additionally, the BLAST algorithm uses an appropriate substitution matrix, nucleotide or amino acid and for gapped alignments uses gap creation and extension penalties. For example, BLAST alignment and comparison of polypeptide sequences are typically done using the BLOSUM62 matrix, a gap existence penalty of 11 and a gap extension penalty of 1.

In some embodiments, sequence similarity scores are reported from BLAST analyses done using the BLOSUM62 matrix, a gap existence penalty of 11 and a gap extension penalty of 1.

In a particular embodiment, sequence identity/similarity scores provided herein refer to the value obtained using GAP Version 10 (GCG, Accelrys, San Diego, Calif.) using the following parameters: % identity and % similarity for a nucleotide sequence using GAP Weight of 50 and Length Weight of 3, and the nwsgapdna.cmp scoring matrix; % identity and similarity for an amino acid sequence using GAP Weight of 8 and Length Weight of 2, and the BLOSUM62 scoring matrix (Henikoff and Henikoff, PNAS USA. 89:10915-10919, 1992). GAP uses the algorithm of Needleman and Wunsch (J Mol Biol. 48:443-453, 1970) to find the alignment of two complete sequences that maximizes the number of matches and minimizes the number of gaps.

In particular embodiments, the variant polypeptide comprises an amino acid sequence that can be optimally aligned with a reference polypeptide sequence (see, e.g., Table U1, Sequence Listing) to generate a BLAST bit scores or sequence similarity scores of at least about 50, 60, 70, 80, 90, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more, including all integers and ranges in between, wherein the BLAST alignment used the BLOSUM62 matrix, a gap existence penalty of 11, and a gap extension penalty of 1.

As noted above, a reference polypeptide may be altered in various ways including amino acid substitutions, deletions, truncations, additions, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (PNAS USA. 82: 488-492, 1985); Kunkel et al., (Methods in Enzymol. 154: 367-382, 1987), U.S. Pat. No. 4,873,192, Watson, J. D. et al., (“Molecular Biology of the Gene,” Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Methods for screening gene products of combinatorial libraries made by such modifications, and for screening cDNA libraries for gene products having a selected property are known in the art. Such methods are adaptable for rapid screening of the gene libraries generated by combinatorial mutagenesis of reference polypeptides. As one example, recursive ensemble mutagenesis (REM), a technique which enhances the frequency of functional mutants in the libraries, can be used in combination with the screening assays to identify polypeptide variants (Arkin and Yourvan, PNAS USA 89: 7811-7815, 1992; Delgrave et al., Protein Engineering. 6: 327-331, 1993).

For in vivo use, the ulinastatin polypeptides and fusion polypeptides described herein are typically formulated as part of a pharmaceutical or therapeutic composition. Certain embodiments thus employ compositions, for example, therapeutic or pharmaceutic compositions, comprising a ulinastatin polypeptide described herein, and a pharmaceutically-acceptable carrier. “Carriers” can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

The compositions may be prepared by methodology well known in the pharmaceutical art. For example, a composition can be prepared by combining a composition that comprises a ulinastatin polypeptide, as described herein, and optionally one or more of buffers or excipients, optionally with sterile, distilled water so as to form a solution. Certain compositions comprise a physiological saline solution (e.g., 0.9% normal saline) or dextrose (e.g., about 1-10% dextrose, or 1, 2, 3, 4, 5, 6, 7, 8, 9, or 10% dextrose). A surfactant can be added to facilitate the formation of a homogeneous solution or suspension. Surfactants are compounds that non-covalently interact with the ulinastatin polypeptide in the composition so as to facilitate dissolution or homogeneous suspension of the polypeptide in the aqueous delivery system.

A pharmaceutical carrier may be liquid, semi-liquid, or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Specific compositions comprise a mature ulinastatin polypeptide with a T17-O-linked glycan, comprising (i) a modified O-linked glycosylation site at residues Glu-Gly-Ser-Gly (SEQ ID NO: 5) which reduces glycosylation at the O-linked glycosylation site, (ii) an N-linked glycan at residue at residue N45, and (iii) an O-linked glycan at residue T17, the residues being defined by SEQ ID NO: 1 or 2, as described herein, and a pharmaceutically-acceptable carrier. Additional examples of mature ulinastatin polypeptides, and active variants and fragments thereof, are described herein (see, for example, Table U1 and related disclosure).

Certain compositions comprise a mixture of ulinastatin glycoforms. For example, certain compositions comprise (a) a first mature ulinastatin polypetide comprising the T17-O-linked glycan, as described herein, and (b) a second mature ulinastatin polypeptide, which comprises the N-linked glycan at residue N45 and does not comprise the O-linked glycan at residue T17. In some embodiments, the second mature ulinastatin polypeptide has a modified O-linked glycosylation site at residues Glu-Gly-Ser-Gly (SEQ ID NO: 5), as described herein, for example, the S10A substitution, which reduces glycosylation at the O-linked glycosylation site. In some embodiments, the second, mature ulinastatin polypeptide of (b) comprises, consists, or consists essentially of an amino acid sequence that is at least 80, 85, 90, 95, 96, 97, 98, 99, or 100% identical to SEQ ID NO: 2 or 4, comprises or retains the modified O-linked glycosylation site (e.g., the S10A substitution) and the N-linked glycan at residue N45, does not comprise the O-linked glycan at residue T17, and has at least one ulinastatin activity. In some embodiments, the mature ulinastatin polypeptides of (a):(b) are present in the composition at a ratio ranging from about 20:1 to about 1:20, optionally about 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, or 1:20.

Certain compositions are substantially pure on a protein basis or weight-basis. For instance, as above, certain compositions have a purity of at least about 80%, 85%, 90%, 95%, 98%, or 99% on a protein basis or a weight-weight basis and are substantially aggregate-free, for example, less than about 10, 9, 8, 7, 6, or 5% aggregated. Certain compositions are substantially endotoxin-free, as described herein. In specific embodiments, the composition has one or more of the following determinations of purity: less than about 1 EU endotoxin/mg protein, less that about 100 ng host cell protein/mg protein, less than about 10 μg host cell DNA/mg protein, and/or is substantially aggregate-free.

Certain compositions are at a pharmaceutically-acceptable pH. For instance, in certain embodiments, the pharmaceutically-acceptable pH is about 5.0 to about 8.0 (±0.01 to ±0.1), or about 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, or 8.0 (±0.01 to ±0.1), including all integers and ranges in between.

In certain embodiments, a ulinastatin polypeptide has at least one ulinastatin activity at a pH close to the physiological pH of human blood. Thus, in some embodiments, a ulinastatin polypeptide has at least one ulinastatin activity at a pH of about 4 to about 10.8, or about 6 to about 8, or about 6.5 to about 7.5. In certain embodiments, a ulinastatin polypeptide has effective ulinastatin activity at about pH 7.4.

Administration may be achieved by a variety of different routes. Modes of administration depend upon the nature of the condition to be treated or prevented. For example, a composition can be administered orally, intranasally, intraperitoneally, parenterally, intravenously, intralymphatically, intratumorly, intramuscularly, interstitially, intraintestinally, intra-arterially, subcutaneously, intraocularly, intrasynovial, transepithelial, and/or transdermally. Particular embodiments include by intravenous (IV), subcutaneous (SC), inhalatory, topical, or oral administration.

Particular embodiments administering the pharmaceutical composition to the subject with a lung disease by inhalatory administration. For instance, certain embodiments include administering with a nebulizer, a metered-dose inhaler, or a dry powdered inhaler.

Specific embodiments comprise administering the pharmaceutical composition to the subject with a skin or mucous membrane disease by topical or oral administration, for example, as an oral mouthwash. For topical administration, the carrier may comprise a solution, emulsion, ointment, or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a therapeutic or pharmaceutical composition for topical administration. If intended for transdermal administration, the composition may include a transdermal patch or iontophoresis device.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

In some embodiments, a dosage is administered from about once a day to about once every two or three weeks. For example, in certain embodiments, a dosage is administered about once every 1, 2, 3, 4, 5, 6, or 7 days, or about once a week, or about twice a week, or about three times a week, or about once every two or three weeks.

Certain embodiments comprise administering a ulinastatin polypeptide at a dosage (e.g., a daily dosage) of about 1×10⁴ U to about 1×10⁵ U to about 100×10⁵ U, or about 1×10⁴ U, 2×10⁴ U, 3×10⁴ U, 4×10⁴ U, 5×10⁴ U, 6×10⁴ U, 7×10⁴ U, 8×10⁴ U, 9×10⁴ U, 1×10⁵ U, 2×10⁵ U, 3×10⁵ U, 4×10⁵ U, 5×10⁵ U, 6×10⁵ U, 7×10⁵ U, 8×10⁵ U, 9×10⁵ U, 10×10⁵ U, 11×10⁵ U, 12×10⁵ U, 15×10⁵ U, 20×10⁵ U, 30×10⁵U, 40×10⁵ U, 50×10⁵ U, 60×10⁵ U, 70×10⁵ U, 80×10⁵ U, or 100×10⁵ U, including all ranges and integers in between. Certain embodiments comprise administering a ulinastatin polypeptide at a dosage (e.g., a daily dosage) of about 50,000 U/kg to about 1,000,000 U/kg, including all integers and ranges in between, for example, a dosage of about 50,000 U/kg, 55,000 U/kg, 60,000 U/kg, 65,000 U/kg, 70,000 U/kg, 75,000 U/kg, 80,000 U/kg, 85,000 U/kg, 90,000 U/kg, 95,000 U/kg, 100,000 U/kg, 115,000 U/kg, 120,000 U/kg, 125,000 U/kg, 130,000 U/kg, 135,000 U/kg, 140,000 U/kg, 145,000 U/kg, 150,000 U/kg, 155,000 U/kg, 160,000 U/kg, 165,000 U/kg, 170,000 U/kg, 175,000 U/kg, 180,000 U/kg, 185,000 U/kg, 190,000 U/kg, 195,000 U/kg, 200,000 U/kg, 200,000 U/kg, 215,000 U/kg, 220,000 U/kg, 225,000 U/kg, 230,000 U/kg, 235,000 U/kg, 240,000 U/kg, 245,000 U/kg, 250,000 U/kg, 255,000 U/kg, 260,000 U/kg, 265,000 U/kg, 270,000 U/kg, 275,000 U/kg, 280,000 U/kg, 285,000 U/kg, 290,000 U/kg, 295,000 U/kg, 300,000 U/kg, 300,000 U/kg, 315,000 U/kg, 320,000 U/kg, 325,000 U/kg, 330,000 U/kg, 335,000 U/kg, 340,000 U/kg, 345,000 U/kg, 350,000 U/kg, 355,000 U/kg, 360,000 U/kg, 365,000 U/kg, 370,000 U/kg, 375,000 U/kg, 380,000 U/kg, 385,000 U/kg, 390,000 U/kg, 395,000 U/kg, 400,000 U/kg, 400,000 U/kg, 415,000 U/kg, 420,000 U/kg, 425,000 U/kg, 430,000 U/kg, 435,000 U/kg, 440,000 U/kg, 445,000 U/kg, 450,000 U/kg, 455,000 U/kg, 460,000 U/kg, 465,000 U/kg, 470,000 U/kg, 475,000 U/kg, 480,000 U/kg, 485,000 U/kg, 490,000 U/kg, 495,000 U/kg, 500,000 U/kg, 500,000 U/kg, 515,000 U/kg, 520,000 U/kg, 525,000 U/kg, 530,000 U/kg, 535,000 U/kg, 540,000 U/kg, 545,000 U/kg, 550,000 U/kg, 555,000 U/kg, 560,000 U/kg, 565,000 U/kg, 570,000 U/kg, 575,000 U/kg, 580,000 U/kg, 585,000 U/kg, 590,000 U/kg, 595,000 U/kg, 600,000 U/kg, 600,000 U/kg, 615,000 U/kg, 620,000 U/kg, 625,000 U/kg, 630,000 U/kg, 635,000 U/kg, 640,000 U/kg, 645,000 U/kg, 650,000 U/kg, 655,000 U/kg, 660,000 U/kg, 665,000 U/kg, 670,000 U/kg, 675,000 U/kg, 680,000 U/kg, 685,000 U/kg, 690,000 U/kg, 695,000 U/kg, 700,000 U/kg, 700,000 U/kg, 715,000 U/kg, 720,000 U/kg, 725,000 U/kg, 730,000 U/kg, 735,000 U/kg, 740,000 U/kg, 745,000 U/kg, 750,000 U/kg, 755,000 U/kg, 760,000 U/kg, 765,000 U/kg, 770,000 U/kg, 775,000 U/kg, 780,000 U/kg, 785,000 U/kg, 790,000 U/kg, 795,000 U/kg, 800,000 U/kg, 800,000 U/kg, 815,000 U/kg, 820,000 U/kg, 825,000 U/kg, 830,000 U/kg, 835,000 U/kg, 840,000 U/kg, 845,000 U/kg, 850,000 U/kg, 855,000 U/kg, 860,000 U/kg, 865,000 U/kg, 870,000 U/kg, 875,000 U/kg, 880,000 U/kg, 885,000 U/kg, 890,000 U/kg, 895,000 U/kg, 900,000 U/kg, 900,000 U/kg, 915,000 U/kg, 920,000 U/kg, 925,000 U/kg, 930,000 U/kg, 935,000 U/kg, 940,000 U/kg, 945,000 U/kg, 950,000 U/kg, 955,000 U/kg, 960,000 U/kg, 965,000 U/kg, 970,000 U/kg, 975,000 U/kg, 980,000 U/kg, 985,000 U/kg, 990,000 U/kg, 995,000 U/kg, or 1,000,000 U/kg or more. In certain instances, treatment is initiated with small dosages which can be increased by small increments until the optimum effect under the circumstances is achieved.

In some embodiments, a therapeutically effective amount or therapeutic dosage of a composition described herein is an amount that is effective to reduce NE levels or activity in a subject and/or improve one or more clinical characteristics of disease. For instance, in some embodiments, administration of a pharmaceutical composition reduces NE levels or activity in the subject by about or at least about 10, 20, 30, 40, 50, 60, 70, 80, 90, 100, 200, 300, 400, 500, 600, 700, 800, 900, 1000% or more relative to a control or reference, for instance, as measured over a period of about 1, 2, 3, 4, 5, 6, 7, 8, 9, 10, 11, 12, 13, 14, 15, 16, 17, 18, 19, 20, 21, 22, 23, or 24 months or more.

In certain embodiments, administration of a pharmaceutical composition increase the life expectancy of the subject in need thereof, for example, by about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more years.

In some embodiments, the subject has a lung disease and administration of the pharmaceutical composition improves respiratory function in the subject by about or at least about 10, 20, 30, 40, 50, 60, 70, 80, 90, 100, 200, 300, 400, 500, 600, 700, 800, 900, 1000% or more relative to a control or reference, for example, as measured over a period of about 1, 2, 3, 4, 5, 6, 7, 8, 9, 10, 11, 12, 13, 14, 15, 16, 17, 18, 19, 20, 21, 22, 23, or 24 months or more. In some instances, the improved respiratory function is selected from one or more of increased expiration time, increased inspiration time, decreased peak expiratory flow, decreased peak inspiratory flow, decreased respiratory minute volume (RMV), and decreased respiratory rate. In particular embodiments, the subject has a skin or mucous membrane disease and administration of the pharmaceutical composition reduces skin or mucous membrane lesions or ulcers in the subject by about or at least about 10, 20, 30, 40, 50, 60, 70, 80, 90, 100, 200, 300, 400, 500, 600, 700, 800, 900, 1000% or more relative to a control or reference, for example, as measured over a period of about 1, 2, 3, 4, 5, 6, 7, 8, 9, 10, 11, 12, 13, 14, 15, 16, 17, 18, 19, 20, 21, 22, 23, or 24 months or more. Methods and assays for measuring such symptoms are known in the art.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Inhibition of Neutrophil Elastase

In vitro studies were performed to test the ability of ulinastatin to inhibit neutrophil elastase (NE). The recombinant ulinastatin polypeptide (DM300) in this experiment is the UTIΔCS variant (SEQ ID NO: 2), which has an N-linked glycan at residue N45 and a non-natural O-linked glycan at residue T17 after recombinant preparation according to methods described in U.S. Provisional Application Nos. 63/108,733 and 63/021,938 (herein incorporated by reference).

Recombinant ulinastatin (DM300) was tested for inhibition of neutrophil elastase at 8 concentrations of DM300, with triplicate measurements per compound concentration. Neutrophil elastase activity was measured using the Neutrophil Elastase Colorimetric Drug Discovery Assay (Enzo Life Sciences) as per the manufacturer's protocol. 65 μl of assay buffer was placed into the required number of wells of a 96 well, clear, half area plate and equilibrated to 37° C. Inhibitor was added to a final reaction concentration at 100, 31.6, 10, 3.16, 1, 0.316, 0.1, 0.0316, or 0 μg/mL DM300 by addition of 20 μl of 5× concentration of DM300 diluted in assay buffer, followed by 10 μl of diluted neutrophil elastase. 100 μM elastatinal was also included as a control. After 15 minutes at 37° C., 5 μl of diluted substrate was added and the plate was read in SpectraMax m5 Microplate Reader equilibrated to 37° C. Linear portions of the data were selected, and the resulting data was analyzed using GraphPad Prism V.8.2.1.

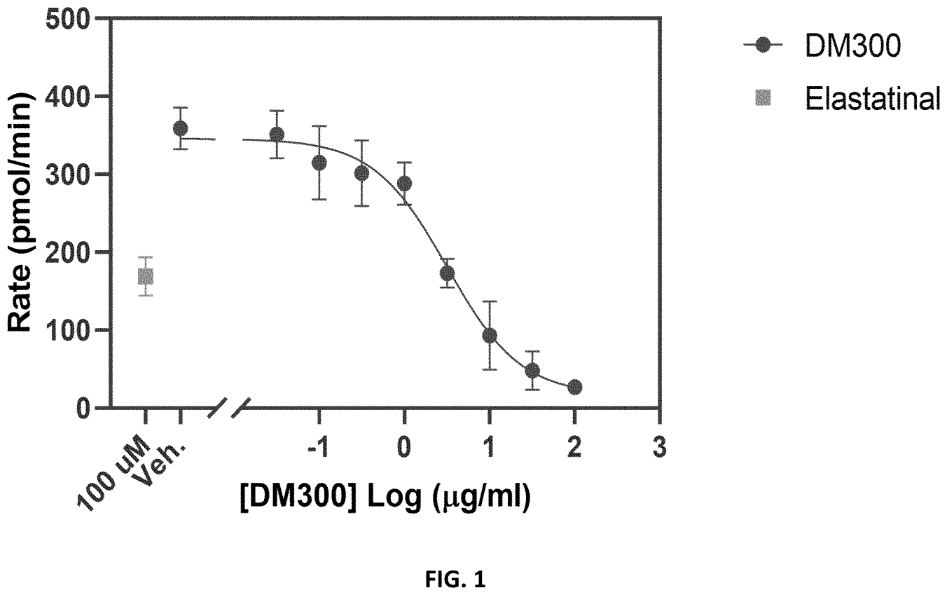

The results are shown in FIG. 1 and Table E1 below.

TABLE E1

| | DM300 | Elastatinal |
|---|---|---|
| log(inhibitor) vs. response - | | Too few points |
| Variable slope (four parameters) | | |
| Best-fit values | | |
| Bottom | 16.65 | |
| Top | 345.8 | |
| LogIC₅₀ | 0.4987 | |
| HillSlope | −1.004 | |
| IC₅₀ | 3.153 μg/ml | |
| Span | 329.1 | |
| 95% CI (profile likelihood) | | |
| Bottom | −64.94 to 56.39 | |
| Top | 321.1 to 378.9 | |
| LogIC₅₀ | 0.2958 to 0.7976 | |
| HillSlope | −1.812 to −0.5688 | |
| IC₅₀ | 1.976 to 6.275 | |
| Goodness of Fit | | |
| Degrees of Freedom | 23 | |
| R squared | 0.9484 | |
| Sum of Squares | 22897 | |
| Sy · x | 31.55 | |
| Number of points | | |
| # of X values | 27 | 30 |
| # Y values analyzed | 27 | 3 |

Under the tested conditions, the IC₅₀ for DM300 was about 3.153 μg/ml or about 43.79 nM (assuming 1 Da=1 g/mol for an approximately 17.2 kDa protein). Recombinant ulinastatin (e.g., DM300) thus has potential therapeutic utility as an inhibitor of neutrophil elastase activity, for example, in the treatment of neutrophil elastase (NE)-associated diseases such as alpha-1 antitrypsin (A1AT) deficiency.

1. A method of treating, ameliorating the symptoms of, or inhibiting the progression of, a neutrophil elastase (NE)-associated disease in a subject in need thereof, comprising administering to the subject a pharmaceutical composition comprising a ulinastatin polypeptide.

2. The method of claim 1, wherein the NE-associated disease comprises a lung disease.

3. The method of claim 1, wherein the lung disease is selected from one or more of alpha-1 antitrypsin (A1AT) deficiency including complications thereof, cystic fibrosis, pneumonia, acute lung injury (ALI), acute respiratory distress syndrome (ARDS), chronic obstructive pulmonary disease (COPD), bronchiectasis, and virus-induced lung injury optionally associated with coronavirus (e.g., Covid-19) or influenza infection, including combinations thereof.

4. The method of claim 3, wherein the NE-associated disease is alpha-1 antitrypsin (Al AT)-deficiency including complications thereof.

5. The method of claim 4, wherein the subject has reduced levels of serum A1AT, optionally about or less than about 80% of normal serum level of A1AT, about or less than about 60% of normal serum level of A1AT, about or less than about 40% of normal serum level of A1AT), or about 10-15% of normal serum level of A1ST; or
wherein the subject has an A1AT phenotype/genotype optionally selected from PiMS, PiSS, PiMZ, PiSZ, and PiZZ, or
wherein the subject has an A1AT genotype selected from a deficiency A1AT allele and a null A1AT allele.

6-7. (canceled)

8. The method of claim 5, wherein the A1AT deficiency allele is an E342K allele (Z allele) or an E264V allele (S allele).

9. The method of claim 4, comprising:
(a) determining serum A1AT levels and/or A1AT phenotype/genotype in the subject; and
(b) administering the pharmaceutical composition to the subject if the subject has an A1AT phenotype/genotype and/or reduced serum levels of A1AT levels relative to normal.

10. The method of claim 9, wherein the A1AT phenotype/genotype is selected from PiMS, PiSS, PiMZ, PiSZ, and PiZZ; wherein the A1AT genotype comprises a deficiency A1AT allele or a null A1AT allele; and/or wherein the serum A1AT levels are about or less than about 80%, 60%, 40%, 20%, 15%, or 10% of normal serum level of A1AT.

11. The method of claim 10, wherein the A1AT deficiency allele is an E342K allele (Z allele) or an E264V allele (S allele).

12. The method of claim 4, wherein the complications of A1AT-deficiency include any one or more of asthma, bronchiectasis, cirrhosis, COPD, respiratory failure, and vasculitis.

13. The method of claim 2, wherein the subject has any one or more of shortness of breath (optionally on exertion and later at rest), wheezing, sputum production, or recurrent respiratory infections.

14. The method of claim 4, comprising administering the pharmaceutical composition comprising the ulinastatin polypeptide in combination with an additional therapeutic agent for treating A1AT-deficiency, optionally an RNA interference (RNAi) agent directed against a disease-associated A1AT mutant, A1AT augmentation therapy, a bronchodilator, an inhaled steroid, an antibiotic agent, or an anti-viral agent.

15. The method of claim 2, wherein the NE-associated disease is cystic fibrosis, and wherein the subject has mutation in the gene cystic fibrosis transmembrane conductance regulator (CFTR).

16. The method of claim 1, wherein the NE-associated disease comprises a skin or mucous membrane disease, optionally wherein the skin or mucous membrane disease is Behcet's Disease, pemphigus vulgaris, pemphigus foliaceus, bullous pemphigoid, and Kawasaki disease.

17. The method of claim le, wherein the NE-associated disease is an autoimmune disease, optionally selected from myasthenia gravis, warm autoimmune hemolytic anemia, thyroid eye disease, idiopathic thrombocytopenic purpura, chronic inflammatory demyelinating polyneuropathy, neuromyelitis optica, Guillain-Barre Syndrome, and PLA2R+membranous nephropathy.

18. The method of claim 1, wherein the subject has increased NE levels or activity relative to a reference standard or healthy control.

19. The method of claim 1, comprising:
(a) determining NE levels or activity in a sample from the subject; and
(b) administering the pharmaceutical composition to the subject if the sample has increased NE levels or activity relative to a reference standard or healthy control.

20. The method of claim 19, wherein the sample is a sputum sample (optionally comprising mucus of the lower airways), a blood or serum sample, or a skin or mucous membrane sample.

21. The method of claim 20, wherein the sputum sample comprises airway neutrophils, and the method comprises determining levels or activity of surface-bound NE in the sample.

22. The method of claim 20, wherein the method comprises determining levels or activity of free NE in the sample.

23. The method of claim 1, wherein the ulinastatin polypeptide is a recombinant ulinastatin polypeptide or a urinary-derived ulinastatin polypeptide.

24. The method of claim 1, wherein the ulinastatin polypeptide comprises, consists, or consists essentially of SEQ ID NO: 2, and comprises an N-linked glycan at residue at residue N45 and an O-linked glycan at residue T17 (DM300).

25. The method of claim 1, wherein the ulinastatin polypeptide comprises, consists, or consists essentially of an amino acid sequence selected from Table U1, or an active variant or fragment thereof that has at least one ulinastatin activity.

26. The method of claim 25, wherein the ulinastatin polypeptide comprises, consists, or consists essentially of:
(a) SEQ ID NO:1 or a variant of SEQ ID NO: 1 that has at least one at least one modification to an O-linked glycosylation and at least one ulinastatin activity;
(b) a fragment of SEQ ID NO: 1 or (a) that has at least one ulinastatin activity; or
(c) a variant of (a) or (b) that is at least 80, 85, 90, 95, 96, 97, 98, or 99% identical to (a) or (b) and has at least one ulinastatin activity.

27. The method of claim 26, wherein (a) comprises a substitution or deletion at the O-linked glycosylation site of residues 8-11 of SEQ ID NO: 1, optionally a substitution or deletion at residue S10 of SEQ ID NO: 1, optionally an S10A substitution, or wherein (b) comprises, consists, or consists essentially of about 20, 25, 30, 35, 40, 45, 50, 55, 60, 65, 70, 75, 80, 85, 90, 95, 100, 105, 110, 115, 120, 125, or 130 contiguous amino acids of SEQ ID NO:1 or 2.

28. The method of claim 26, wherein (b) comprises, consists, or consists essentially of about 20-130, 20-120, 20-110, 20-100, 20-90, 20-80, 20-70, 20-60, 20-50, 20-40, 20-30, 30-130, 30-120, 30-110, 30-100, 30-90, 30-80, 30-70, 30-60, 30-50, 30-40, 40-130, 40-120, 40-110, 40-100, 40-90, 40-80, 40-70, 40-60, 40-50, 50-130, 50-120, 50-110, 50-100, 50-90, 50-80, 50-70, 50-60, 60-130, 60-120, 60-110, 60-100, 60-90, 60-80, 60-70, 70-130, 70-120, 70-110, 70-100, 70-90, 70-80, 80-130, 80-120, 80-110, 80-100, 80-90, 90-130, 90-120, 90-110, 90-100, 100-130, 100-120, or 100-110 contiguous amino acids of SEQ ID NO: 1 or 2.

29-30. (canceled)

31. The method of claim 26, wherein the ulinastatin polypeptide comprises, consists, or consists essentially of an amino acid sequence that is at least 80, 85, 90, 95, 96, 97, 98, 99, or 100% identical to SEQ ID NO: 2 (UTIΔCS), which retains a substitution at position S10 of SEQ ID NO: 2, optionally a S10A substitution.

32. The method of claim 31, wherein the ulinastatin polypeptide has an N-linked glycan at residue at residue N45 and an O-linked glycan at residue T17, the residues being defined by SEQ ID NO: 1 or 2.

33. The method of claim 1, wherein the ulinastatin polypeptide is fused to an Fc region, to form a ulinastatin-Fc fusion polypeptide, wherein the ulinastatin-Fc fusion polypeptide has at least one ulinastatin activity.

34. The method of claim 33, wherein the Fc region comprises, consists, or consists essentially of CH2 region, CH3 region, CH4 region, and/or hinge region(s) from a IgA, IgD, IgE, IgG, or IgM immunoglobulin heavy chain, or
wherein the Fc region comprises, consists, or consists essentially of one or more of the human Fc region amino acid sequences of Table F1, including variants, fragments, homologs, orthologs, paralogs, and combinations thereof, or
wherein the ulinastatin-Fc fusion polypeptide comprises a modified Fc region which has at least one altered effector function and/or pharmacokinetic (PK) characteristic relative to a wild-type Fc region.

35-36. (canceled)

37. The method of claim 1, wherein the at least one ulinastatin activity comprises a protease inhibitor activity, optionally a neutrophil elastase (NE)-inhibitor activity or a trypsin inhibitor activity, and optionally an anti-inflammatory activity.

38. The method of claim 37, wherein the at least one ulinastatin activity is a trypsin inhibitor activity, and wherein the ulinastatin polypeptide has a specific activity as a trypsin inhibitor of about or at least about 1000-3000 U/mg, or about or at least about 1000, 1100, 1200, 1300, 1400, 1500, 1600, 1700, 1800, 1900, 2000, 2100, 2200, 2300, 2400, 2500, 2600, 2700, 2800, 2900, or 3000 U/mg, wherein one unit (U) is an amount of the ulinastatin polypeptide that inhibits the activity of 2 μg trypsin by 50%, or
wherein the at least one ulinastatin activity is an NE-inhibitor activity, and
wherein the ulinastatin polypeptide has an IC₅₀ in an in vitro assay of neutrophil elastase activity of about 10 μg/ml or lower value, optionally an IC₅₀ of about 10, 9, 8, 7, 6, 5, 4, 3, 2, or 1 μg/ml or lower value, including all ranges and integers in between, optionally an IC₅₀ of about 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-9, 3-7, 3-6, 3-5, 3-4 10 μg/ml; or
wherein the ulinastatin polypeptide has an IC₅₀ in an in vitro assay of neutrophil elastase activity of about 100 nM or lower value, optionally an IC₅₀ of about 100, 90, 80, 70, 60, 50, 40, 30, 20, or 10 nM or lower value, including all ranges and integers in between, optionally an IC₅₀ of about 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, or 40-50 nM.

39-41. (canceled)

42. The method of claim 1, comprising administering the pharmaceutical composition to the subject by intravenous (IV), subcutaneous (SC), inhalatory, topical, or oral administration.

43. The method of claim 42, comprising administering the pharmaceutical composition to the subject with a lung disease by inhalatory administration, optionally with a nebulizer, a metered-dose inhaler, or a dry powdered inhaler, or
comprising administering the pharmaceutical composition to the subject with a skin or mucous membrane disease by topical or oral administration, optionally as an oral mouthwash.

44. (canceled)

45. The method of claim 1, wherein the pharmaceutical composition comprises the ulinastatin polypeptide at a dosage of about 50,000 U/kg to about 1,000,000 U/kg, including all integers and ranges in between.

46. The method of claim 1, wherein administration of the pharmaceutical composition reduces NE levels or activity in the subject, optionally by about or at least about 10, 20, 30, 40, 50, 60, 70, 80, 90, 100, 200, 300, 400, 500, 600, 700, 800, 900, 1000% or more relative to a control or reference, optionally as measured over a period of about 1, 2, 3, 4, 5, 6, 7, 8, 9, 10, 11, 12, 13, 14, 15, 16, 17, 18, 19, 20, 21, 22, 23, or 24 months or more.

47. The method of claim 1, wherein administration of the pharmaceutical composition increase the life expectancy of the subject in need thereof, optionally by about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more years.

48. The method of claim 1, wherein the subject has a lung disease and administration of the pharmaceutical composition improves respiratory function in the subject by about or at least about 10, 20, 30, 40, 50, 60, 70, 80, 90, 100, 200, 300, 400, 500, 600, 700, 800, 900, 1000% or more relative to a control or reference, optionally as measured over a period of about 1, 2, 3, 4, 5, 6, 7, 8, 9, 10, 11, 12, 13, 14, 15, 16, 17, 18, 19, 20, 21, 22, 23, or 24 months or more.

49. The method of claim 48, wherein the improved respiratory function is selected from one or more of increased expiration time, increased inspiration time, decreased peak expiratory flow, decreased peak inspiratory flow, decreased respiratory minute volume (RMV), and decreased respiratory rate.

50. The method of claim 1, wherein the subject has a skin or mucous membrane disease and administration of the pharmaceutical composition reduces skin or mucous membrane lesions or ulcers in the subject by about or at least about 10, 20, 30, 40, 50, 60, 70, 80, 90, 100, 200, 300, 400, 500, 600, 700, 800, 900, 1000% or more relative to a control or reference, optionally as measured over a period of about 1, 2, 3, 4, 5, 6, 7, 8, 9, 10, 11, 12, 13, 14, 15, 16, 17, 18, 19, 20, 21, 22, 23, or 24 months or more.
